(19) United States
(12) Patent Application Publication
(10) Pub. No.: US 2024/0000496 A1
(43) Pub. Date: Jan. 4, 2024

(54) SECONDARY ELECTRODE ASSEMBLY FOR TISSUE CONTACT DETERMINATIONS

(71) Applicant: Cook Medical Technologies LLC, Bloomington, IN (US)

(72) Inventor: Vincent McHugo, Birdhill (IE)

(73) Assignee: Cook Medical Technologies LLC, Bloomington, IN (US)

(21) Appl. No.: 18/215,613

(22) Filed: Jun. 28, 2023

Related U.S. Application Data
(60) Provisional application No. 63/356,586, filed on Jun. 29, 2022.

(51) Int. Cl. A61B 18/12

(52) U.S. Cl. CPC A61B 18/1233; A61B 2018/00875

(57) ABSTRACT

A medical device includes: a secondary electrode assembly comprising a plurality of periphery secondary electrode sets configured to sense a plurality of localized impedances for a periphery of a primary electrode assembly. Additionally, a controller is configured to: permit delivery of electrosurgical energy to the primary electrode assembly in response to a determination that the primary electrode assembly is sufficiently contacting the tissue based on the plurality of localized impedances sensed by the secondary electrode assembly.

### CROSS-REFERENCE TO RELATED APPLICATIONS

The present application claims the benefit of priority to U.S. Provisional Application No. 63/356,586, filed Jun. 29, 2022. The contents of U.S. Provisional Application No. 63/356,586 are herein incorporated by reference in their entirety.

### TECHNICAL FIELD

The present invention relates generally to medical devices, and more particularly to electrode assemblies for electrosurgical medical devices.

### BACKGROUND

Electrosurgical medical devices include a conductive element that applies electrosurgical energy to tissue at a treatment site of a patient to cause a desired electrosurgical effect on the tissue, such as ablation, coagulation, or cutting. To achieve the desired electrosurgical effect, the conductive element may apply the electrosurgical energy according to a desired energy density, which in turn may be achieved by ensuring that the conductive element is sufficiently in contact with the tissue. Conversely, if the conductive element is not in sufficient contact with the tissue when applying the electrosurgical energy, then the desired electrosurgical effect on the tissue may not be achieved. As such, ways to optimally detect whether the conductive element is in sufficient contact with the tissue may be desirable.

### BRIEF SUMMARY

The present description describes electrosurgical systems, electrosurgical devices, electrode systems, and related methods that include a secondary electrode assembly configured to sense localized impedances for a periphery of a primary electrode assembly. In one embodiment, an electrosurgical device includes: an elongate member extending from a proximal portion to a distal portion; a primary electrode assembly at the distal portion, the primary electrode assembly configured to contact tissue for performance of an electrosurgical procedure; and a secondary electrode assembly comprising a plurality of periphery secondary electrode sets, each periphery secondary electrode set configured to sense a respective one of a plurality of localized impedances for a periphery of the primary electrode assembly.

In another embodiment, an electrosurgical system includes: a controller configured to: receive at least one signal indicative of a plurality of localized impedances for a periphery of a primary electrode assembly configured to cause an electrosurgical effect on tissue at a treatment site within a patient, the plurality of localized impedances sensed by a secondary electrode assembly; and permit delivery of electrosurgical energy to the primary electrode assembly in response to a determination that the primary electrode assembly is sufficiently contacting the tissue based on the plurality of localized impedances sensed by the secondary electrode assembly.

In another embodiment, a method for electrosurgery includes: moving a distal portion of an electrosurgical device to a treatment site within a patient, the distal portion comprising a primary electrode assembly and a secondary electrode assembly; while the primary electrode assembly is deactivated, activating the secondary electrode assembly; in response to activating the secondary electrode assembly, measuring, with a controller, a plurality of localized impedances for a periphery of the primary electrode assembly; determining, with the controller, that the primary electrode assembly is in sufficient contact with tissue at the treatment site based on the plurality of localized impedances; and in response to determining that the primary electrode assembly is in sufficient contact with the tissue at the treatment site, activating the primary electrode assembly with electrosurgical energy to cause an electrosurgical effect on the tissue.

Other embodiments are possible, and each of the embodiments can be used alone or together in combination. Accordingly, various embodiments will now be described with reference to the attached drawings.

### BRIEF DESCRIPTION OF THE DRAWINGS

FIG. 1 shows a diagram of an electrosurgical system including an electrosurgical device and a power source.

FIG. 2A shows an example configuration of a distal portion of the electrosurgical device of FIG. 1, including an electrode system coupled to a distal end of an elongate member, with the electrode system configured in a retracted state.

FIG. 2B shows the distal portion of FIG. 2A, with the electrode system configured in an expanded state.

FIG. 3A shows an example configuration of a primary electrode assembly and a secondary electrode assembly for the electrode system of FIGS. 1-2B.

FIG. 3B shows another example configuration of a primary electrode assembly and a secondary electrode assembly for the electrode system of FIGS. 1-2B.

FIG. 3C shows a third example configuration of a primary electrode assembly and a secondary electrode assembly for the electrode system of FIGS. 1-2B.

FIG. 3D shows a fourth example configuration for a primary electrode assembly and a secondary electrode assembly for the electrode system of FIG. 1-2B.

FIG. 4 shows an example configuration for a controller of the electrosurgical system of FIG. 1.

FIG. 5A shows an example configuration for a secondary electrode assembly to communicate with a controller over an elongate member of the electrosurgical device of FIG. 1 using a single transmission line.

FIG. 5B shows another example configuration for a secondary electrode assembly to communicate with a controller over an elongate member of the electrosurgical device of FIG. 1 using a single transmission line.

FIG. 5C shows a third example configuration for a secondary electrode assembly to communicate with a controller over an elongate member of the electrosurgical device of FIG. 1 using a single transmission line.

FIG. 5D shows a fourth example configuration for a secondary electrode assembly to communicate with a controller over an elongate member of the electrosurgical device of FIG. 1 using a single transmission line.

FIG. 6 shows a flow chart of an example method of operating the electrosurgical device of FIG. 1.

FIG. 7 shows a flow chart of another example method of operating the electrosurgical device of FIG. 1.

### DETAILED DESCRIPTION

The present description describes various embodiments of an electrode system, electrosurgical medical devices and electrosurgical medical systems that include an electrode system, and related methods for performance of an electrosurgical procedure and/or operating an electrosurgical device. The electrode system, and related electrosurgical systems, devices, and methods, may include a primary electrode system and a secondary electrode system. The secondary electrode system may sense localized impedances for a periphery and/or a core of the primary electrode system. Based on the localized impedances, a determination of whether the primary electrode assembly is sufficiently contacting tissue at a treatment site may be made.

Determining whether the primary electrode assembly is sufficiently contacting tissue may avoid or minimize situations where electrosurgical energy is delivered to the primary electrode assembly when the primary electrode assembly is not sufficiently contacting tissue. When the primary electrode assembly is sufficiently contacting tissue, the primary electrode assembly may cause a desired or intended electrosurgical effect on tissue in response to receipt of electrosurgical energy according to a desired current or energy density. On the other hand, if the primary electrode assembly is not sufficiently contacting tissue when receiving electrosurgical energy, then the primary electrode assembly may transfer the electrosurgical energy to the tissue without a desired current or energy density, which in turn may not cause the desired or intended electrosurgical effect on the tissue, and/or may damage the tissue. As such, determining that the primary electrode assembly is sufficiently contacting the tissue may increase the likelihood of successful electrosurgical operations and minimize harm or damage to tissue.

The electrode system of the present description may differ from other electrode systems that use the same electrode assembly to both deliver electrosurgical energy to cause an electrosurgical effect on tissue and sense impedance to determine if the electrode assembly is sufficiently contacting tissue. Such other electrode systems may sense a single impedance for the electrode assembly. This single impedance may not provide an optimally accurate indication or depiction of the electrode assembly's contact with tissue. For example, the impedance value sensed by the electrode assembly may indicate that the electrode assembly is sufficiently contacting tissue, but in actuality, a certain portion or portions of the electrode assembly is not contacting tissue. Repositioning the electrode assembly, even slightly, may increase the amount of the electrode assembly in contact with the tissue, which in turn may cause the electrode assembly to deliver the electrosurgical energy to the tissue with a current or energy density closer to an optimal current or energy density.

As described herein, the secondary electrode assembly is configured to sense a plurality of localized impedances for a periphery of the primary electrode assembly. These localized impedances may provide a better determination and/or indication of how much of the primary electrode assembly is actually contacting tissue compared to configurations that determine a single impedance using the same electrode assembly for both impedance sensing and transfer of electrosurgical energy for causing an electrosurgical effect. The secondary electrode assembly may include secondary electrode sets positioned near different parts of the primary electrode assembly. The electrosurgical devices may determine which of those secondary electrode sets are, and are not, in contact with the tissue, which may correspondingly provide an indication of parts or sections of the primary electrode assembly that are, and are not, in contact with tissue. In effect, use of the multiple secondary electrode sets, separate from the primary electrode assembly, to sense impedance for different parts or portions of the primary electrode assembly provides a more granular configuration for sensing impedance, which can provide a better indication of an amount of the primary electrode assembly that is in contact with tissue, and/or an indication of which parts or sections of the primary electrode assembly are, and are not, in contact with tissue.

In addition, sensing impedance with the multiple secondary electrode sets may provide an indication of how to reposition the distal portion of the electrosurgical device. For example, by sensing impedance of the multiple secondary electrode sets, an operator can determine which of the electrode sets are, and are not, in contact with tissue. By obtaining this information, an operator can determine if he/she needs to reposition the distal portion of the electrosurgical device to obtain better contact with tissue, and if so, how to move the distal portion in order to obtain the better contact. To illustrate, if a secondary electrode set indicates than an upper left corner of the primary electrode assembly is not in contact with tissue, the operator may use that information to determine how to move the distal portion at the treatment site in order to have the upper left corner or region of the primary electrode assembly contact the tissue.

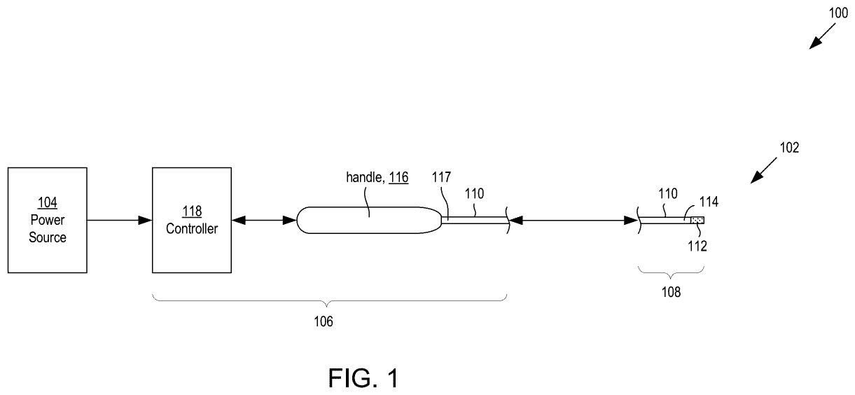

FIG. 1 shows a diagram of an example electrosurgical system 100 that includes an electrosurgical device 102 coupled to a power source 104. The power source 104 is configured to generate and output electrosurgical energy or power. The electrosurgical device 102, by being coupled to the power source 104, may be configured to receive the electrosurgical energy from the power source 104, and supply or deliver the electrosurgical energy to tissue at a treatment site, such as a treatment site within a patient. The electrosurgical device 102, and/or the electrosurgical system 100 including the electrosurgical device 102 and the power source 104, may be configured to perform an electrosurgical procedure, during which the electrosurgical device 102, through application of the electrosurgical energy, is configured to cause a desired or intended electrosurgical effect on the tissue at the treatment site. Example electrosurgical effects include ablation, coagulation, or cutting, as non-limiting examples.

The electrosurgical energy that the power source 104 generates and that the electrosurgical device 102 delivers to the treatment site may include or have any of various electrical properties or characteristics to cause the electrosurgical effect on the tissue. For example, the electrosurgical energy may have an associated energy level (such as in Joules) or an associated power level (such as in Watts) at which the power source 104 generates the electrosurgical energy. In addition or alternatively, the electrosurgical energy may include an electrical current that travels through the electrical device 102 to the treatment site. The electrical current itself may have or be defined by any of various electrical properties or characteristics with associated values that, in turn, may characterize, quantify, or otherwise determine an associated energy level or power level of the electrosurgical energy being delivered to the treatment site. For example, the electrical current may have an associated amplitude. In addition or alternatively, the electrical current may be an alternating current (AC) with an associated frequency. In particular examples, the electrical current is an AC current having a frequency in the radio frequency (RF) range, and/or in the kiloHertz (kHz) or MegaHertz (MHz) range. Other examples where the electrical current is an AC current with a frequency lower than kHz range (e.g., <1 kHz) or higher than the MHz range (e.g., >999 MHz), such as in the GigaHertz (GHz) or TeraHertz (THz) a direct current (DC), or where the electrical current is a direct current (DC) may be possible.

The electrosurgical device 102 may longitudinally extend from a proximal portion 106 to a distal portion 108. In general, the proximal portion 106 may be a portion of the electrosurgical device 102, and/or may include components of the electrosurgical device 102, that remains outside of the patient during an electrosurgical operation or procedure. The distal portion 108 may be a portion of the electrosurgical device 102, and/or may include components of the electrosurgical device 102, that is delivered to the treatment site, and remains at the treatment site and contacts the tissue at the treatment site during an electrosurgical operation or procedure.

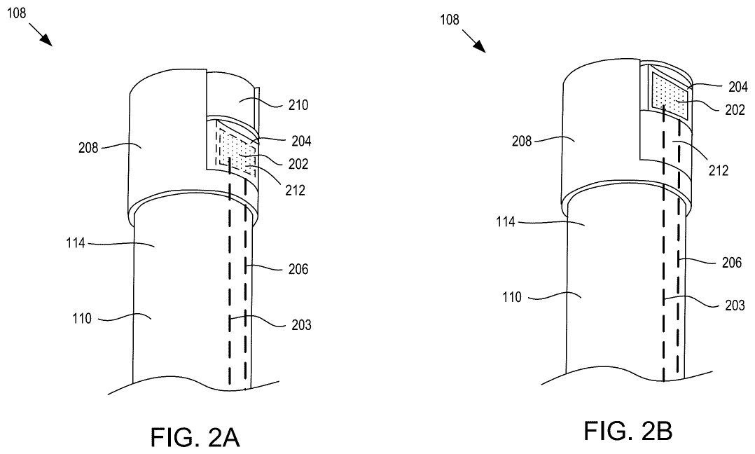

Additionally, the electrosurgical device 102 may include an elongate member 110 that longitudinally extends from the proximal portion 106 to the distal portion 108. The elongate member 110 may be in the form of any of various elongate structures suitable for insertion into a patient. For example, in some embodiments, the elongate member 110 may be a solid structure, such as a rod. In other embodiments, the elongate member 110 may be a tubular structure, such as by having one or lumens and/or channels longitudinally extending through a body of the tubular structure. As non-limiting examples, the elongate member 110 may be an endoscope or a catheter configured for insertion into and movable within a lumen or channel of an endoscope. Various configuration of the elongate member 110 may be possible. Also, the distal portion 108 may include an electrode system 112 that forms at least part of and/or that is coupled to a distal end 114 of the elongate member 110. As described in further detail below with reference to FIGS. 2A, 2B, the electrode system 112 may include one or more electrodes configured to contact tissue and/or deliver electrosurgical energy to the tissue.

Also, for at least some configurations, the electrode system 112 may be configurable in and/or movable between a deployed state (such as an expanded state) and an undeployed state (such as a retracted state). In the deployed state, the electrode system 112 be configured to and/or able to contact the tissue at the treatment site. In the undeployed state, the electrode system may be unable to contact the tissue at the treatment site.

The proximal portion 106 of the electrosurgical device 102 may include a handle 116 that an operator can grasp to control movement of the electrosurgical device 102. For example, the handle 116 may facilitate movement of the distal portion 108 to the treatment site. In addition or alternatively, the handle 116 may control movement, including longitudinal and/or axial rotational movement, of the elongate member 110 and/or the distal portion 108 of the electrosurgical device 102, including the electrode system 112. Through control of the movement, the handle 116 may be configured to move the electrode system 112 to optimally position it at the treatment site. In addition or alternatively, the handle 116 may be configured to control movement of the electrode system 112 between deployed and undeployed states. For example, although not shown in FIG. 1, a control wire or other control mechanism may longitudinally extend through or alongside the elongate member 110 and be operably coupled to the handle 116 and the electrode system 112. The handle 116 may be configured to control movement of the control wire, and in turn movement of the electrode system 112 between the deployed and undeployed states. As shown in FIG. 1, the handle 116 is coupled to a proximal end 117

In addition, the proximal portion 106 may include a controller 118 configured to control delivery of electrosurgical energy from the power source 104 to the electrosurgical device 102 and correspondingly, to the treatment site. Accordingly, as shown in FIG. 1, the controller 118 may be positioned between the power source 104 and the handle 116, such that electrosurgical energy passes through the controller 118 in order to reach the handle 116. In configurations other than the one shown in FIG. 1, the controller 118 may part of the power source 104 (such as by being an internal component of the power source 104), or may be a component of the electrosurgical system 100 separate from the electrosurgical device 102 and the power source 104. Accordingly, for at least some embodiments, the electrosurgical device 102 may not include the controller 118.

FIGS. 2A and 2B show an example configuration of the distal end 108 of the electrosurgical device 102, illustrating an example configuration of the electrode system 112 in more detail. FIG. 2A shows the electrode system 112 in a retracted state and FIG. 2B shows the electrode system 112 in an expanded state.

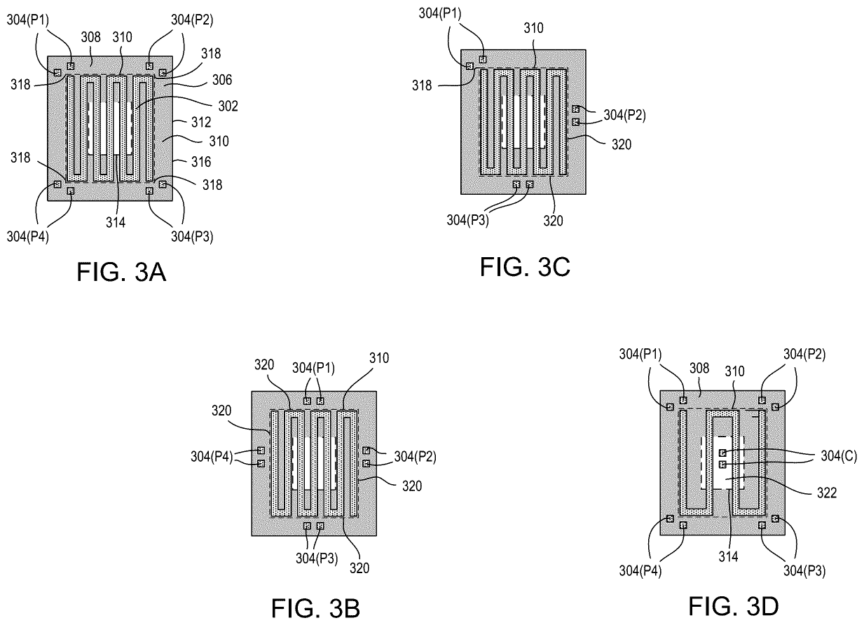

With reference to either or both FIGS. 2A and 2B, the electrode system 112 includes at least one electrode 202. In various configurations of the electrosurgical device 102, the at least one electrode 202 may include one or more electrodes of one or more different types. A first type of electrode may be configured to deliver or transfer electrosurgical energy to the tissue it is contacting in order to cause an electrosurgical effect on the tissue, such as ablation, coagulation, or cutting, as non-limiting examples. A second type of electrode may be configured to sense one or more characteristics of treatment site, such as impedance or environmental temperature, as non-limiting examples. Additionally, although FIGS. 2A and 2B show the at least one electrode 202 generically as a box or rectangle shape, the at least one electrode 202 may have any of various shapes or structural configurations that will allow it to contact tissue for an intended electrosurgical procedure in accordance with the one or more electrode types. As described in further detail below with reference to FIGS. 3A-3D, the at least one electrode 202 may include a primary electrode assembly that includes one or more electrodes configured as a first type, and a secondary electrode assembly that includes one or more electrodes configured as a second type.

Also, as shown in FIGS. 2A and 2B, the at least one electrode 202 may be coupled to, including electrically coupled to, at least one elongate conductive element 203, such as one or more conductive wires for example, longitudinally extending with the elongate member 110 between the proximal portion 106 and the distal portion 108. The at least one elongate conductive element 203 may be configured to deliver or transfer electrosurgical energy and/or electrical current to the at least one electrode 202. Also, for at least some embodiments as described in further detail below, the at least one elongate conductive element 203 may include at least one transmission line configured to transmit or communicate one or more electrical signals, such as one or more signals indicative of one or more sensed impedances and/or one or more signals carrying electrode contact results of one or more electrode assemblies.

In addition, for at least some configurations, the electrode system 112 may include at least one substrate, carrier, or other support structure 204 on which the at least one electrode 202 is mounted, supported, and/or disposed. The substrate 204 may be made of a non-conductive or insulating material. For at least some configurations where the at least one electrode 202 includes multiple electrodes, the at least substrate 204 may include only one substrate, such that the multiple electrodes are disposed on the same or a common substrate. In other configurations, the at least one substrate 204 includes multiple substrates, such that different electrodes may be disposed on different substrates. Various configurations utilizing one or more substrates for mounting one or more electrodes are possible.

Also, for configurations where the at least one electrode 202 is configured to move between retracted and expanded configurations, the at least one substrate 204 may be configured to move with the at least one electrode 204. For at least some configurations such as shown in FIGS. 2A and 2B, movement of the at least one electrode 202 and the at least one substrate 204 may be controlled by a control wire 206 longitudinally extending with the elongate member 110 and operatively coupled to the at least one substrate 204 and the handle 116. Accordingly, the handle 116 may control movement of the control wire 206, which in turn controls movement of the at least one substrate 204 and the at least one electrode 202. For example, the handle 116 may cause distal movement of the control wire 206, which in turn may cause distal movement of the at least one electrode 202 and the at least one substrate 204, such as to move the electrode system 112 from the retracted state to the expanded state. Also, the handle 116 may cause proximal movement of the control wire 206, which in turn may cause proximal movement of the at least one electrode 202 and the at least one substrate 204, such as to move the electrode system 112 from the expanded state to the retracted state.

The electrode system 112 may also include a support structure or base 208 that provides structural support for the at least one electrode 202 and the at least one substrate 204. The support structure 208 may also be configured to guide movement, such as by providing a track 210 through or over which the at least one electrode 202 and the at least one substrate 204 is configured to longitudinally move. For at least some configurations such as shown in FIGS. 2A and 2B, the support structure 208 may be in the form of a cap or other cylindrical element coupled to the distal end 114 of the elongate member 110. For other example configurations, the support structure 208, and/or the electrode system 112 as a whole, may be an integral part of the distal end 114 of the elongate member 110.

Additionally, the support structure 208 may include a housing or cover 212 for the at least one electrode 202 when the at least one electrode is in the retracted state. As described, the at least one electrode 202 is unable to contact tissue at the treatment site in the retracted state. Accordingly, as shown in FIG. 2A, when the at least one electrode 202 is in the retracted state, the cover 212 functions to cover the at least one electrode 202 and/or act as a barrier between the at least one electrode 202 and the tissue so as to prevent the at least one electrode 202 from contacting the tissue. When moving from the retracted to the expanded state, the at least one electrode 202 may distally move past the cover 212 and be exposed to its outer surroundings, so as to be able to contact the tissue. FIG. 2B shows the at least one electrode 202 positioned distally past the cover 212 and configured to contact tissue.

The configuration of the electrode system 112 is merely exemplary and other configurations may be possible. For example, in other configurations, the at least one electrode 202 may be in a fixed position—i.e., not movable between retracted and expanded states—and/or may be configured to always be exposed to outer surroundings so as to be able to contact tissue.

FIGS. 3A-3D show example configurations for the at least one electrode 202 and the at least one substrate 204 of FIGS. 2A and 2B. Referring to FIG. 3A, the at least one electrode 202 may include a primary electrode assembly 302 and a secondary electrode assembly 304. Also, in the example configuration shown in FIG. 3A, the primary and secondary electrode assemblies 302, 304 are configured or disposed on a single or same substrate 306. In other configurations, the primary and secondary electrode assemblies 302, 304 may be disposed on multiple substrates. For example, the primary electrode assembly 302 may be disposed on one substrate, and the secondary electrode assembly 304 may be disposed on another substrate. As another example, the primary electrode assembly 302 and the secondary electrode assembly 304 may each be disposed on multiple substrates. In another example, the primary electrode assembly 302 may be on only one substrate, and different parts of the secondary electrode assembly 304 may be on different substrates. In another example, one part of the primary electrode assembly 302 and one part of the secondary electrode assembly 304 may be on one substrate, and a second part of the primary electrode assembly 302 and a second part of the secondary electrode assembly 304 may be on a second substrate. Various ways of configuring the primary and secondary electrode assemblies 302, 304 on one or more substrates may be possible.

Additionally, the primary electrode assembly 302 is configured to deliver or transmit electrosurgical energy to tissue at a treatment site to cause an electrosurgical effect on tissue, such as ablation, coagulation, or cutting as examples. For example, the primary electrode assembly 302 may be configured as the first electrode type, as previously described. Additionally, as previously described, the electrosurgical energy to cause the electrosurgical effect may be generated by the power source 104 and transferred to the electrosurgical device 102, including through the controller 118, the handle 116, through a transmission line of the at least one conductive element 203 longitudinally extending through or alongside the elongate member 110, and to the primary electrode assembly 302. The primary electrode assembly 302 may be considered activated upon receipt of the electrosurgical energy and/or when it is conducting electrical current of the electrosurgical energy.

In addition, in any of various configurations, the primary electrode assembly 302 may be configured to have a monopolar configuration or a bipolar configuration. In general, a complete electrical path for electrical energy delivered to the treatment site may include an active path and a return path. The active path may include at least one active electrode, and the return path may include at least one return electrode. For a monopolar configuration, the active path and/or its active electrode are integrated with the electrosurgical device, and the return path and/or its return electrode are separate or remote from the electrosurgical device. For example, the return electrode may be positioned outside of the patient, such as by being affixed to the patient's skin of an appendage (e.g., a leg), and the rest of the return path may extend outside of the patient from the return electrode back to the power source 104. With respect to the electrosurgical device 102, for a monopolar configuration, the one or more active electrodes of the active path are part of the electrode system 112, the at least one electrode 202, and/or the primary electrode assembly 302, and the one or more return electrodes of the return path may be separate or remote from the electrode system 112, the at least one electrode 202, and/or the primary electrode assembly 302.

For a bipolar configuration, both the active path and its active electrode(s) and the return path and its return electrode(s) are integrated with the electrosurgical device. Accordingly, for a bipolar configuration, both the active path and the return path may extend while the electrosurgical device is positioned within a patient for performance an electrosurgical operation. With respect to the electrosurgical device 102, for a bipolar configuration, the one or more active electrodes of the active path and the one or more return electrodes of the return path both are part of the electrode system 112, the at least one electrode 202, and/or the primary electrode assembly 302.

In the example configurations shown in FIGS. 3A-3D, the primary electrode assembly 302 has a monopolar configuration, where the primary electrode assembly 302 is configured as a single, contiguous conductive element. For these configurations, the entirety of the primary electrode assembly 302 is configured to be electrically activated at the same time or electrically deactivated at the same time. In other words, the primary electrode assembly 302 does not have multiple conductive elements electrically isolated from each other that can be independently electrically activated from each other. In other configurations, the primary electrode assembly 302 may have a monopolar configuration and include multiple conductive elements electrically isolated from one another and that can be independently electrically activated from each other. For example, the primary electrode assembly 302 may include multiple conductive elements that are configured to cause electrosurgical effects on different areas or parts of the tissue. Such multiple conductive elements may be configured to cause the electrosurgical effects on the different parts of the tissue according to any of various timing schemes, such as simultaneously, during different but overlapping time periods, or during different and non-overlapping time periods (such as sequentially or in an alternating manner). In still other configurations, the primary electrode assembly 302 may have a bipolar configuration that includes multiple conductive elements electrically isolated from each other, where one or more of the conductive elements are part of an active path and/or form one or more active electrodes, and one or more other conductive elements are part of a return path and/or form one or more return electrodes. Various ways of configuring the primary electrode assembly 302 as a single conductive element or multiple electrically isolated conductive elements and/or in a monopolar or a bipolar manner may be possible.

Also, in the example configuration in FIG. 3A, the primary electrode assembly 302 is configured to have a snake or serpentine shape. The electrode shape of the primary electrode assembly 302 shown in FIG. 3A is merely exemplary, and any of various other shapes suitable for causing an electrosurgical effect on tissue may be possible.

The secondary electrode assembly 304 is configured to sense at least one characteristic of the treatment site, such as impedance or temperature. For example, the secondary electrode 304 may include one or more electrodes of the second electrode type, as previously described. Accordingly, in general, the secondary electrode assembly 304 is used for one or more functions different from delivering electrosurgical energy to tissue to cause an electrosurgical effect on the tissue. In particular example configurations, the secondary electrode assembly 304 is activated with different electrical energy or power than the electrosurgical energy with which the primary electrode assembly 302 is activated. The different electrical energy may be characterized by a different voltage level, a different current level, a different waveform, a different frequency, a different power level or energy level. In addition or alternatively, the electrical energy used to activate the secondary electrode assembly 304 may include a DC voltage and/or a DC current. In addition or alternatively, the electrical energy used to activate the secondary electrode assembly 304 is a low power signal relative to the electrosurgical energy provide to the primary electrode assembly 302, in that it has a lower power level than the power level of the electrosurgical energy delivered or conducted by the primary electrode assembly 302.

Additionally, the primary and secondary electrode assemblies 302, 304 are electrically isolated from each other, and/or may be configured to be electrically activated and deactivated independent from each other. For example, when the primary electrode assembly 302 is activated and transferring electrosurgical energy, the secondary electrode assembly 304 may be deactivated, as by not conducting electrical current. Similarly, when the secondary electrode assembly 304 is activated, the primary electrode assembly 302 may be deactivated. In addition or alternatively, by being electrically isolated from each other and/or by being configured to be activated independent of each other, the primary and secondary electrode assemblies 302, 304 may be configured to receive, conduct, transfer, and/or be activated by their different electrosurgical and electrical energies, respectively, whether that occurs simultaneously or during different time periods.

The primary and secondary electrode assemblies 302, 304 may be electrically isolated from each other and/or configured to be electrically activated and deactivated independent of each other in any of various ways. For example, as shown in FIG. 3A, the primary and secondary electrode assemblies 302, 304 are spaced apart from each other on the substrate 306. Also, in some configurations, the elongate conductive member 203 may include multiple conductive elements that are separately or independently coupled to the primary and secondary electrode assemblies 302, 304. Such multiple conductive elements may be configured to deliver the electrosurgical energy for activation of the primary electrode assembly 302 and the electrical energy for activation of the secondary electrode assembly 304 in parallel. In other configurations, the same conductive element of the elongate conductive member 203 may be used to deliver the electrosurgical energy for activating the primary electrode assembly 302 and the electrical energy for activating the secondary electrode assembly 304. For such configurations, the distal portion 108 of the electrosurgical device 102 may include a switching circuit (not shown) configured to selectively route the energy it is receiving to the primary electrode assembly 302 or the secondary electrode assembly 304. For example, during a first time period, the switching circuit may be configured to receive electrical energy from the elongate conductive member 203 and route the electrical energy to the secondary electrode assembly 304 while the primary electrode assembly 302 is deactivated. During a second time period, the switching circuit may be configured to receive electrosurgical energy from the elongate conductive member 203 and route the electrosurgical energy to the primary electrode assembly 302 while the secondary electrode assembly 304 is deactivated. Various ways of electrically isolating the primary and secondary electrode assemblies 302, 304 from each other, and/or configuring the primary and secondary electrode assemblies 302, 304 to operate and/or be activated and deactivated independent of each other through separate electrical connections and/or switching circuit configurations may be possible.

Additionally, for some configurations, an overall surface area of the primary electrode assembly 302 is larger than an overall surface area of the secondary electrode assembly 304. For at least some of these configurations, the overall surface area of the primary electrode assembly 302 is at least twice (two times larger than) the overall surface area of the secondary electrode assembly 304. In particular configurations, the overall surface area of the primary electrode assembly 302 is at least ten times the overall surface area of the secondary electrode assembly 304.

In addition, for the example configuration shown in FIG. 3A, the secondary electrode assembly 304 includes a plurality or an N-number (where N is two or more) of periphery secondary electrode sets 304(P) configured to sense a plurality of localized impedances for a periphery, identified by shaded area 308, of the primary electrode assembly 302. The configuration in FIG. 3A has four periphery secondary electrode sets 304(P1), 304(P2), 304(P3), 304(P4), although numbers other than four may be possible. For example, the number of periphery secondary electrode sets 304(P) may include two, three, or five or more.

The periphery 308 of the primary electrode assembly 302 may be defined according to or relative to an outer boundary, identified by dotted line 310, of the primary electrode assembly 302, and may include an outer boundary 312 and an inner boundary 314. The outer boundary 312 may be, or be defined by, an outer edge 316 of the substrate 306. The inner boundary 314 may be a collection of points, where each point is about half the distance between a geometric center point or centroid of the primary electrode assembly 302 and the outer boundary 310.

Each localized impedance may be an impedance for an associated portion or part of the periphery 308. Correspondingly, each of the periphery secondary electrode sets 304(P) may be configured to sense a respective one of the plurality of localized impedances for their associated portions of the periphery 308. For at least some configurations, the periphery secondary electrode sets 304(P) are disposed in the portions of the periphery 308 for which they are to sense their respective localized impedances. Additionally, different periphery electrode sets 304(P) may be configured to sense different localized impedances for different portions of the periphery 308.

As shown in FIG. 3A, each periphery secondary electrode set 304(P) may include a pair of electrodes. A localized impedance that a given ith periphery secondary electrode set 304(Pi) is configured to sense is an impedance between the pair of electrodes. The sensed localized impedance may be, or may be proportional to, an amount of current that flows between the pair of electrodes for a given voltage level applied across the pair of electrodes. Additionally, a value of the localized impedance may be indicative of whether the ith periphery secondary electrode set 304(Pi) is in contact with tissue. That is, if the ith periphery secondary electrode set 304(Pi) is in contact with tissue, then the value of the localized impedance that the ith periphery secondary electrode set 304(Pi) senses may be, or may correspond to, the impedance value of the tissue the ith periphery secondary electrode set 304(Pi) is contacting, which may be a relatively low value below 1 kiloOhm (kΩ), such as below 300 Ohms (Ω) for example. On the other hand, if the ith periphery secondary electrode set 304(Pi) is not in contact with tissue, then the value of the localized impedance that the ith periphery secondary electrode set 304(Pi) senses may be, or may correspond to, an open circuit, which is a value much larger than the tissue impedance, such as in the kΩ or MegaOhm (MΩ) range.

In addition, the outer boundary 310 is generally a boundary line that tracks or identifies an outer perimeter and/or contour of the primary electrode assembly 302. Accordingly, the shape of the outer boundary 310 depends on the shape and configuration of the primary electrode assembly. For at least some configurations, the outer boundary may have a polygonal shape. For example, the shape may be rectangular, as shown in FIG. 3A. Other shapes, including other polygonal shapes (triangular, pentagonal, hexagonal, octagonal, etc.), or other non-polygonal shapes (circular, elliptical, amorphous, etc.) may be possible.

Additionally, each of the periphery secondary electrode sets 304(P) may have an associated position relative to the outer boundary 310 of the primary electrode assembly 302. For at least some example configurations, at least one of the periphery secondary electrode sets 304(P) is disposed outside of the outer boundary 310. For at least some of these configurations such as in FIG. 3A, all of the periphery secondary electrode sets 304(P) are disposed outside of the outer boundary 310. In other example configurations, at least one of the periphery secondary electrode sets may be disposed within or inside of the outer boundary 310.

In addition, for at least some example configurations such as shown in FIG. 3A, at least one of the periphery secondary electrode sets 304(P) is disposed adjacent to a corner 318 of the outer boundary 310. For at least some of these configurations, at least two of the periphery secondary electrode sets 304(P) are disposed adjacent to at least two corners 318 of the outer boundary 310. In particular of these configurations, each of the periphery secondary electrode sets 304(P) is disposed adjacent to a respective or different one of the corners 318. For example, as shown in FIG. 3A, each of the four periphery secondary electrode sets 304(P) is disposed adjacent to a respective or different one of the four corners 318 of the outer boundary 310.

In other example configurations, at least one of the periphery secondary electrode sets 304(P) is disposed adjacent to a side of the outer boundary 310. For at least some of these configurations, at least two of the periphery secondary electrode sets 304(P) are disposed adjacent to at least two sides of the outer boundary 310. In particular of these configurations, each of the periphery secondary electrode sets 304(P) is disposed adjacent to a respective or different one of the sides. For example, FIG. 3B shows an example configuration that is similar to the configuration of FIG. 3A, except that instead of being disposed adjacent to the corners 318, each of the four periphery secondary electrode sets 304(P1), 304(P2), 304(P3), 304(P4) is disposed adjacent to a respective or different one of four sides 320 of the outer boundary 308.

In still other example configurations, at least one of the periphery secondary electrode sets 304(P) is disposed adjacent to a corner 318 of the outer boundary 310 and at least one other of the periphery secondary electrode sets 304(P) is disposed adjacent to a side 320 of the outer boundary 310. For example, FIG. 3C shows an example configuration that includes one periphery secondary electrode set 304(P1) disposed adjacent to a corner 318, and two periphery secondary electrode sets 304(P2), 304(P3) disposed adjacent to respective sides 320.

FIG. 3D shows another example configuration for the at least one electrode 202 and the at least one substrate 204 of FIGS. 2A and 2B, where in addition to the periphery secondary electrode sets 304(P), the secondary electrode assembly 304 further includes a core secondary electrode set 304(C) configured to sense a localized impedance for a core or core area 322 of the primary electrode assembly 302. As shown in FIG. 3D, the core 322 may be an area inside of the periphery 308 and/or within the inner boundary 314. The core secondary electrode set 304(C) may be disposed within the core 322.

Referring back to FIG. 1, the controller 118 may be configured to receive at least one electrical signal indicative of a plurality of localized impedances sensed by the secondary electrode assembly 304 for at least one of the periphery 308 or the core 322 of the primary electrode assembly 302. The electrical signal may include electrical current and/or voltage, amounts of which may indicate the localized impedances sensed by the secondary electrode assembly 304, and/or that can be used by the controller 118 to determine impedance values for the localized impedances. For at least some configurations, in response to receipt of the at least one signal, the controller 118 may control delivery of electrosurgical energy to the primary electrode assembly 302 in response to a determination of whether the primary electrode assembly 302 is sufficiently contacting the tissue based on the plurality of localized impedances sensed by the secondary electrode assembly 304. For example, based on the localized impedances, if a determination is made that the primary electrode assembly 302 is sufficiently contacting tissue, then the controller 118 may permit delivery of electrosurgical energy to the primary electrode assembly 302. In addition, based on the localized impedances, if a determination is made that the primary electrode assembly 302 is not sufficiently contacting tissue, then the controller 118 may prevent delivery of electrosurgical energy to the primary electrode assembly 302. For some configurations, the determination of whether the primary electrode assembly 302 is sufficiently contacting tissue may be made internally by the controller 118. In other configurations, the determination of whether the primary electrode assembly 302 is sufficiently contacting tissue may be made externally, such as by a computing device external to the controller 118, the electrosurgical device 102, and/or the electrosurgical system 100 as a whole, or by a person, such as an operator of the electrosurgical device 102. For such other configurations, the controller 118 may be configured to output the localized impedances or information indicating whether the secondary electrode sets are in contact with tissue, that may enable the external computing device or the person to determine whether the primary electrode assembly 302 is sufficiently contacting tissue.

Additionally, in some configurations, the controller 118 may have direct, internal, and/or automatic control of the delivery of electro surgical energy to the primary electrode assembly 302 based on the sensed localized impedances. For example, the controller 118 itself may determine whether the primary electrode assembly 302 is sufficiently contacting tissue based on the localized impedances, and in response to the determination, permit the electrosurgical energy to be delivered to the primary electrode assembly 302, or prevent the electrosurgical energy from being delivered to the primary electrode assembly 302. In other configurations, the controller 118 may have indirect control of the electrosurgical energy to the primary electrode assembly 302 based on the sensed localized impedances. For example, an external computing device may determine whether the primary electrode assembly 302 is sufficiently contacting tissue based on the sensed localized impedances, and send a control signal to the controller 118 based on the determination, that controls the controller 118 whether to permit electrosurgical energy to the primary electrode assembly 302 or prevent the electrosurgical energy from being delivered to the primary electrode assembly 302. As another example, a person may determine whether the primary electrode assembly 302 is sufficiently contacting tissue based on the sensed localized impedances, and operate or manipulate a manual control on the controller 118 that controls the controller 118 whether to permit electrosurgical energy to the primary electrode assembly 302 or prevent the electrosurgical energy from being delivered to the primary electrode assembly 302.

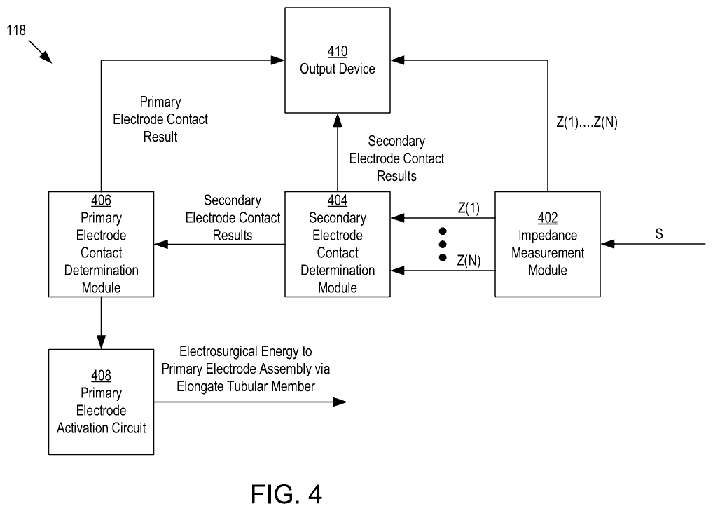

FIG. 4 shows a block diagram of an example configuration of the controller 118 in further detail. As shown in FIG. 4, the controller 118 may include one or more modules. As used herein, a module may be hardware or a combination of hardware and software. For example, a module may include an application specific integrated circuit (ASIC), a field programmable gate array (FPGA), a circuit, a digital logic circuit, an analog circuit, a combination of discrete circuits, gates, or any other type of hardware or any of various combinations thereof. In addition or alternatively, each module may include memory hardware that comprises instructions executable with a processor or processor circuitry to implement one or more of the features of the module. When any one of the module includes the portion of the memory that comprises instructions executable with the processor, the module may or may not include the processor. In some examples, each module may just be the portion of the memory that comprises instructions executable with the processor to implement the features of the corresponding module without the module including any other hardware. Because each module includes at least some hardware even when the included hardware comprises software, each module may be interchangeably referred to as a hardware module.

As shown in FIG. 4, the controller 118 may include an impedance measurement module 402 configured to determine or measure impedance values for the localized impedances sensed by the secondary electrode assembly 304. The impedance measurement module 402 may be configured to determine the impedance values in any of various ways. For example, the impedance measurement module 402 may be configured to receive at least one electrical signal S that indicates the plurality of localized impedances, and measure the impedance values based on the at least one signal S. The at least one electrical signal S may include electrical signals output by the secondary electrode sets of the secondary electrode assembly 304, and/or may include electrical currents or otherwise indicate current amounts of electrical currents drawn through each of the secondary electrode sets. Accordingly, based on the at least one signal S, the impedance measurement module 402 may be configured to determine the amount of electrical current drawn through each of the secondary electrode sets. The impedance measurement module 402 may also determine a voltage level applied to each of the secondary electrode sets to cause the current draw, in turn determine a localized impedance Z for each of the secondary electrode sets.

As shown in FIG. 4, the impedance measurement module 402 may determine an N-number of localized impedances Z(1)-Z(N) for an N-number of secondary electrode sets. The N-number of secondary electrode sets may include an N-number of periphery secondary electrode sets 304(P), or a Q-number of periphery secondary electrode sets 304(P) and a R-number of core secondary electrode sets 304(C), where N, Q, and R are each integers, N=Q+R, N is two or more, Q is two or more, and R is one or more.

For at least some configurations, the impedance measurement module 402 may determine the N-number of localized impedances Z(1)-Z(N) individually, sequentially, or otherwise at different times from each other. For example, the impedance measurement module 402 may measure the localized impedances Z(1)-Z(N) individually for each of the N-number of secondary electrode sets in a predetermined sequential order until all of the localized impedances Z(1)-Z(N) are measured. Determining the localized impedances individually, sequentially, and/or at different times, as opposed to simultaneously, may prevent interference between the localized impedance measurements, which in turn may provide more accurate measurements.

In addition, for at least some configurations, the controller 118 may include a secondary electrode contact determination module 404 that is configured to determine secondary electrode contact results for the secondary electrode sets of the secondary electrode assembly 304. In general, a contact result is a determination and/or an indication of whether an electrode, an electrode set, or an electrode assembly is in contact with tissue. The secondary electrode contact determination module 404 may determine contact results for each of the secondary electrode sets based on the localized impedances Z determined by the impedance measurement module 402. For example, as shown in FIG. 4, the secondary electrode contact determination module 404 may receive an N-number of localized impedance Z(1)-Z(N) for an N-number of secondary electrode sets from the impedance measurement module 402. In response, the secondary electrode contact determination module 404 may determine contact results for each of the N-number secondary electrode sets.

For at least some configurations, the secondary electrode contact determination module 404 may determine secondary electrode contact results by comparing each of the localized impedances Z with an impedance threshold value. For a given ith localized impedance Z(i) for a given ith secondary electrode set, if the value of the ith localized impedance Z(i) is below the impedance threshold value, then the secondary electrode contact determination module 404 may determine that the ith secondary electrode set it in contact with, or sufficiently in contact with, tissue. Additionally, if the value of the ith localized impedance Z(i) is above the impedance threshold value, then the secondary electrode determination module 404 may determine that the ith secondary electrode set is not in contact with, or insufficiently in contact with, tissue.

In addition, for at least some configurations, the controller 118 may include a primary electrode contact determination module 406 that is configured to determine a primary electrode contact result for the primary electrode assembly 302. A primary electrode contact result is a determination and/or an indication of whether the primary electrode assembly 302 is in contact with, or sufficiently in contact with, tissue. The primary electrode contact determination module 406 may determine the primary electrode contact result based on the secondary electrode contact results. For example, the primary electrode contact determination module 406 may receive the secondary electrode contact results from the secondary electrode contact determination module 404. In response, the primary electrode contact determination module 406 may determine the primary contact result for the primary electrode assembly 302.

For at least some configurations, the primary electrode contact determination module 406 may determine the primary electrode contact result based on a number of the secondary electrode contact results indicating that their respective secondary electrode sets are in contact with tissue. Based on the number, the primary electrode contact determination module 406 may determine whether at least a predetermined number of secondary electrode sets is in contact with tissue. For example, if the number of secondary electrode contact results indicating that their respective secondary electrode sets are in contact with tissue is greater than or equal to the predetermined number, then the primary electrode contact determination module 406 may determine that at least the predetermined number of secondary electrode sets is in contact, or in sufficient contact, with tissue. In addition, if the number of secondary electrode contact results indicating that their respective secondary electrode sets are in contact with tissue is less than the predetermined number, then the primary electrode contact determination module 406 may determine that at least the predetermined number of secondary electrode sets is not in contact, or in sufficient contact, with tissue.

Further, if the primary electrode contact determination module 406 determines that at least the predetermined number of secondary electrode sets is in contact, or in sufficient contact, with tissue, then the primary electrode contact determination module 406 may determine that the primary electrode assembly 302 is in contact, or in sufficient contact, with tissue. Additionally, if the primary electrode contact determination module 406 determines that at least the predetermined number of secondary electrode sets is not in contact, or sufficient contact, with tissue, then the primary electrode contact determination module 406 may determine that the primary electrode assembly 302 is not in contact, or in sufficient contact, with tissue.

In addition, for at least some configurations, the controller 118 may include a primary electrode activation circuit 408 that is configured to control activation of the primary electrode assembly 302. The primary electrode activation circuit 408 may control activation of the primary electrode assembly 302 by controlling whether to permit delivery of electrosurgical energy to the primary electrode assembly 302, such as via the handle 116 and/or the elongate member 110. For at least some example configurations, the primary electrode activation circuit 404 may include one or more switches or relays that is configured to selectively permit or prevent the delivery of electrosurgical energy to the primary electrode assembly 302. For example, the one or more switches or relays may be configured in a closed state that permits electrosurgical energy received from the power source 104 to be delivered to the primary electrode assembly 302, and may be configured in an open state that prevents electrosurgical energy from being delivered to the primary electrode assembly 302. In addition or alternatively, the primary electrode activation circuit 408 may include generation circuitry of the power source 104 that is configured to generate the electrosurgical energy. The generation circuitry may permit delivery of the electrosurgical energy to the primary electrode assembly 302 by being activated to generate the electrosurgical energy, and may prevent delivery of the electrosurgical energy to the primary electrode assembly 302 by being deactivated, and therefor unable, to generate the electrosurgical energy.

For at least some example configurations, the primary electrode contact determination module 406 may be configured to control the primary electrode activation circuit 408. In particular, if the primary electrode contact determination module 406 determines that the primary electrode assembly 302 is in contact with, or in sufficient contact with, tissue, then the primary electrode contact determination module 406 may control the primary electrode activation circuit 408 to permit the delivery of electrosurgical energy to the primary electrode assembly 302, and in turn activate the primary electrode assembly 302. In addition, if the primary electrode contact determination module 406 determines that the primary electrode assembly 302 is not in contact with, or not in sufficient contact with, tissue, then the primary electrode contact determination module 406 may control the primary electrode activation circuit 408 to prevent the delivery of electrosurgical energy to the primary electrode assembly 302.

In addition, for at least some configurations, the controller 118 may include, or be coupled to, an output device 410 configured to output at least one of output signal to a person, such as an operator of the electrosurgical system 100. The at least output signal may include one or more of any of various types of signals that can be perceived by the person, such as a visual signal, an audio signal, or a tactile signal, as examples. The output device 410 may include one or more devices, including one or more electronic devices, configured to output the one or more signals. For example, the at least one signal may include a video signal, and the output device 410 may include a display configured to display the video signal. In addition or alternatively, the at least one signal may include an audio signal, and the output device 410 may include an audio speaker configured to audibly output the audio signal. In addition or alternatively, the at least one output signal may include a tactile signal such as a vibrational signal, and the output device 410 may include a device, such as a vibration motor, configured to generate the tactile signal. In addition or alternatively, the at least one output signal may include one or more light signals, and the output device 410 may include one or more light sources (e.g., one or more light emitting diodes (LEDs) as a non-limiting example) configured to output the one or more light signals. Other types of output signals may be possible for the at least one output signal, and/or other types of devices configured to output the at least one output signal may be possible.

The output device 410 may be configured to output information indicating at least one of: the localized impedance values Z determined by the impedance measurement module 402, the secondary electrode contact results determined by the secondary electrode contact determination module 404, or the primary electrode contact result determined by the primary electrode contact determination module 406. The output information may be in the form of the one or more output signals that the output device 410 is configured to output. As non-limiting examples, the output device 410 may include a display configured to display, such as on a display screen, the localized impedance values Z(1) to Z(N); the secondary electrode contact results such as by displaying which of the secondary electrode sets are in contact with tissue and which of the secondary electrode sets are not in contact with tissue, and/or a number of the secondary electrode sets in contact with and/or not in contact with tissue; and/or the primary electrode contact result such as by displaying text or graphics indicating whether or not the primary electrode assembly 302 is determined to be in contact with, or sufficient contact with, tissue. For example, if the primary electrode contact determination module 406 determines that the primary electrode assembly 302 is not in contact with, or sufficient contact with, tissue, then the output device 410 may output an error or warning message indicating that the primary electrode assembly 302 is not contacting, or sufficiently contacting, tissue. Such an error or warning message may further include instructions, or otherwise indicate to an operator, to re-position the electrode system 112 in order to obtain sufficient contact with the tissue. Also, in the event that the primary electrode contact determination module 406 determines that the primary electrode assembly 302 is not in sufficient contact with tissue, displaying which of the secondary electrode sets are and are not in contact with tissue may facilitate to an operator how to re-position the electrode system 112 at the treatment site in order to obtain sufficient contact. As another non-limiting example, the output device 410 may include an N-number of LEDs, and each LED may provide a visual indication of whether an associated secondary electrode set is in contact with, or in sufficient contact with, tissue. For example, an ith LED corresponding to an ith secondary electrode set may turn on and/or light up a first color (e.g., green) to indicate that the ith secondary electrode set is in contact with tissue, and may turn off or light up a second color (e.g., red) to indicate that the ith secondary electrode set is not in contact with tissue. As mentioned, the use of a display or LEDs are merely examples, and any of various other types of output devices to output information to a user to indicate at least one of localized impedance values Z, secondary electrode contact results, or a primary electrode contact result may be possible.

Also, for at least some configurations, the output device 410 may include a network connector that allows the controller 118 to connect to and/or communicate over one or more of any of various types of computer networks, such as a local area network (LAN), a wide area network (WAN), the Internet, a metropolitan area network (MAN), an Internet area network (IAN), a personal area network (PAN), a campus area network (CAN), as non-limiting examples. The network connector may be configured to connect to a computer network via a wired connection or wirelessly. By having a network connector, the output device 410 may be configured to communicate at least one of the determined localized impedance values Z, secondary electrode contact results, or a primary electrode contact device to a remote computing device, such as another computer device or a cloud computing network that is remote from the controller 118, the electrosurgical device 102, and/or the electrosurgical system 100.

Additionally, in any of various configurations, the controller 118 may include all or fewer than all of the components shown in FIG. 4. For example, in some configurations, the controller 118 may be configured without an output device 410 such that the controller 118 is unable to output information indicating any of the localized impedances Z, secondary electrode contact results, and/or primary electrode contact results. In other configurations, the controller 118 is configured without a primary electrode activation circuit 408. For such configurations, the controller 118 may be an electronic device that determines information indicating at least one of localized impedances Z, secondary electrode contact results, or primary electrode contact results, and outputs the information to a person and/or a remote computing device using output device 410, without using the information to control the delivery of electrosurgical energy.

In other configurations, the controller 118 may include the primary electrode activation circuit 408, but does not directly control, or have direct control of, the delivery of electrosurgical energy to the primary electrode assembly 302 based on a primary electrode contact result. By having such direct control, the controller 118 internally determines a primary electrode contact result (such as with the primary electrode contact determination module 406), and controls the primary electrode activation circuit 408 based on the primary electrode contact result without human intervention. For at least some configurations where the controller 118 does not have direct control, the controller 118 may still have indirect control of the delivery of electrosurgical energy to the primary electrode assembly 302. For such configurations, the controller 118 may include a manual control, such as in the form of a button, switch, lever, or voice-activated device as non-limiting examples, that allows a person (for example an operator of the electrosurgical device 102) to manually control the primary electrode activation circuit 408. For example, the manual control may configure the primary electrode activation circuit 408 to prevent the delivery of electrosurgical energy to the primary electrode assembly 302. The controller 118 may output, via the output device 410, information indicating at least one of the localized impedance values Z, the secondary electrode contact results, or the primary electrode contact result, to the person. In response, the person may determine whether the primary electrode assembly 302 is in sufficient contact with tissue. For example, based on the information output by the output device 410, if the person determines that the primary electrode assembly 302 is in sufficient contact with tissue, then the person may manipulate the manual control (such as by pressing or otherwise actuating a foot switch, a button, moving the position of the switch or lever, or audibly providing a command as non-limiting examples) that configures the primary electrode activation circuit 408 to permit electrosurgical energy to the primary electrode assembly 302. On the other hand, if the person determines that the primary electrode assembly 302 is not in sufficient contact with the tissue, then the person may not manipulate the manual control, such that the primary electrode activation circuit 408 continues to be configured to prevent electrosurgical energy from being delivered to the primary electrode assembly 302.

In other configurations, the controller 118 just has the impedance measurement module 402 without the secondary electrode contact determination module 404 and the primary electrode contact determination module 406, or has the impedance measurement module 402 and the secondary electrode contact determination module 404 without the primary electrode contact determination module 406. For such configurations, the controller 118 may output the localized impedances Z and/or the secondary electrode contact results, using the output device 410. In response, a person, such as an operator operating the electrosurgical device 102, may determine whether or which secondary electrode sets are in contact with tissue and/or whether the primary electrode assembly 302 is sufficiently in contact with tissue based on the localized impedances Z and/or the secondary electrode contact results. Based on the determination, the person may determine next steps, such as whether to permit delivery of the electrosurgical energy to the primary electrode assembly 302, such as through manipulation of the manual control as previously described.

Any of various configurations for the controller 118 that includes some or all of the components shown in FIG. 4, and that allows electrosurgical energy to be permitted to be, or prevented from being, delivered to the primary electrode assembly 302 based on localized impedances sensed by the secondary electrode assembly 304 may be possible.

In addition, the controller 118 may determine at least one of the localized impedances Z, the secondary electrode contact results, and/or the primary electrode contact result before any electrosurgical energy is delivered to the primary electrode assembly 302. For example, the determination of whether the primary electrode assembly 302 is sufficiently contacting tissue may be made as part of an initial test procedure and that functions as a prerequisite for whether the controller 118 permits delivery of electrosurgical energy to the primary electrode assembly 302. For example, as long as the controller 118 identifies that the primary electrode assembly 302 is not sufficiently contacting tissue, the controller 118 may be configured to prevent delivery of electrosurgical energy to the primary electrode assembly.

In addition or alternatively, the controller 118 may be configured to determine at least one of the localized impedances Z, the secondary electrode contact results, and/or the primary electrode contact result during delivery of electrosurgical energy. For example, the controller 118 may first determine that the primary electrode assembly 302 is sufficiently contacting tissue, and in response, permit delivery of electrosurgical energy to the primary electrode assembly 302. Then, while the electrosurgical energy is being delivered, the controller 118 may continue to determine at least one of the localized impedance Z, the secondary electrode contact results, and/or the primary electrode contact result. For at least some of the configurations, if the controller 118 determines that the primary electrode assembly 302 is not or no longer sufficiently contacting tissue, the controller 118 may respond by preventing further delivery of electrosurgical energy to the primary electrode assembly. This way, the controller 118 can detect if the primary electrode assembly 302 stops sufficiently contacting tissue during delivery of electrosurgical energy, such as because it moves during the procedure, and in response, prevent further activation of the primary electrode assembly. For at least some of these configurations, the primary electrode assembly 302 and the secondary electrode assembly 304 may be positioned sufficient far away from each other in order to prevent or minimize electrical and/or electromagnetic (EM) interference between the two electrode assemblies 302, 304 and/or the transmission lines to which they are connected.

Additionally, the secondary electrode sets, including the periphery secondary electrode sets 304(P) and/or the core secondary electrode set 304(C), may be configured to communicate electrical signals indicative of the sensed localized impedances to the controller 118 in any of various ways. For some configurations, the secondary electrode sets are configured to independently transmit their sensed localized impedances over the elongate member 110. For such configurations, the at least one elongate conductive element 203 (FIGS. 2A, 2B), may include an N-number of transmission lines, each configured to communicate an electrical signal indicating a localized impedance sensed by an associated secondary electrode set to which the transmission line is coupled. In other configurations, the electrosurgical device 102 is configured to communicate electrical signals indicating sensed localized impedances for the secondary electrode sets, secondary electrode contact results, or a primary electrode contact result over a single or common transmission line. Such other configurations may reduce or minimize the number of transmission lines longitudinally extending within or along the elongate member 110, compared to configurations where the multiple secondary electrode sets separately communicate with the controller 118 over the elongate member 110.

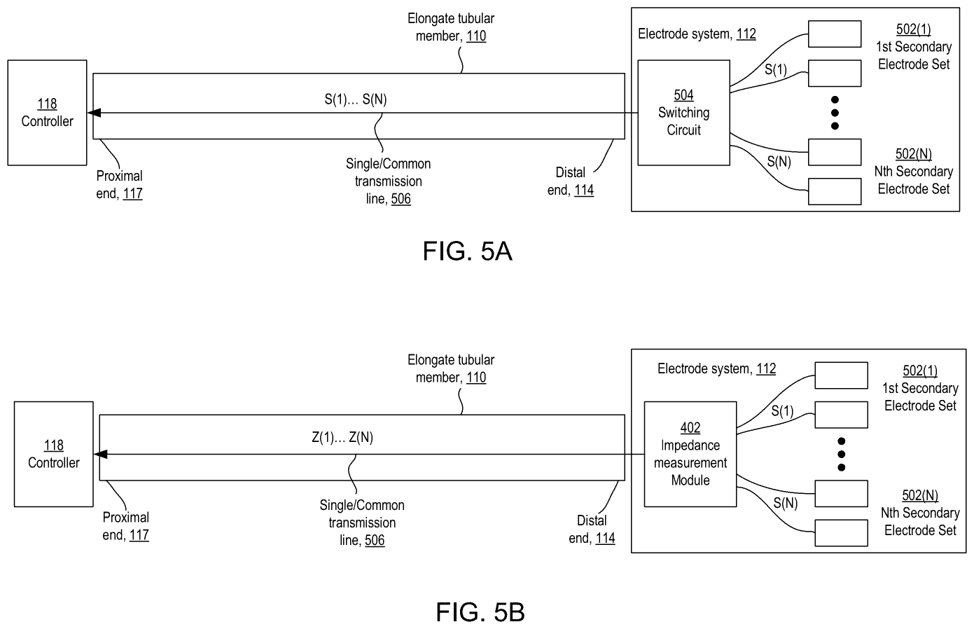

FIGS. 5A-5D show example configurations of the electrosurgical system 100, where the secondary electrode sets communicate with the controller 118 over a single or common transmission line. As shown in FIGS. 5A-4D, the electrosurgical system 112 includes an N-number of secondary electrode sets 502, including a first secondary electrode set 502(1) extending to an Nth secondary electrode set 502(N), where N is two or more. The N-number of secondary electrode sets 502 may include a plurality of periphery secondary electrode sets, such as the plurality of periphery secondary electrode sets 304(P) shown in FIGS. 3A-3C, or a plurality of periphery secondary electrode sets and at least one core secondary electrode set, such as the plurality of periphery secondary electrode sets 304(P) and the core secondary electrode set 304(C) shown in FIG. 3D. Also, as shown in FIGS. 5A-5D, each of the secondary electrode sets 502(1)-502(N) is configured to output a respective electrical signal S(1)-S(N) indicating a sensed localized impedance. Additionally, for simplicity, the primary electrode assembly 302 is not shown in FIGS. 5A-5D, but is otherwise part of the electrode system 112, such as shown in FIGS. 5A-5D.

Referring particularly to FIG. 5A, the electrode system 112 further includes a switching circuit 504 configured to selectively or alternatingly activate each of the secondary electrode sets 502. When an ith secondary electrode set 502(i) is activated by the switching circuit 504, the ith secondary electrode set 502(i) may output or transmit, via the switching circuit 504, an ith electrical signal S(i) over a single or common transmission line 506 that longitudinally extends from the distal end 114 to the proximal end 117 of the elongate member 110 to the controller 118. In this way, the same single/common transmission line 506 communicates or carries the N-number of electrical signals S(1)-S(N) generated by the N-number or secondary electrode sets 502(1)-502(N) to the controller 118. In this context, the electrical signals S(1)-S(N) may include separate, multiple signals, or may include a single signal that carries information indicative of each of the localized impedances sensed by the secondary electrode sets 502(1)-502(N). In response, the controller 118 may process the electrical signals S(1)-S(N) and take further action as previously described with reference to FIG. 4.

FIGS. 5B-5D show other configurations where one or more parts, portions, or components of the controller 118 is included as part of the distal portion 108, such as part of the electrode system 112, instead of as part of the proximal portion 106, proximal the elongate member 110. For the configuration in FIG. 5B, the impedance measurement module 402 is included as part of distal portion 108, such as part of the electrode system 112. The impedance measurement module 402 may receive the signals S(1)-S(N) from the secondary electrode sets 502 at the distal portion 108, and in response, determine localized impedance values Z(1) to Z(N) and transmit the localized impedance values Z(1) to Z(N) over a single or common transmission line 506 to remaining components of the controller 118 positioned proximal the elongate member 118. In response to receipt of the localized impedance values Z(1)-Z(N), the remaining components of the controller 118 may process the localized impedance values Z(1)-Z(N) and take further action as previously described with reference to FIG. 4.

For the configuration in FIG. 5C, the impedance measurement module 402 and the secondary electrode contact determination module 404 are included as part of the distal portion 108, such as part of the electrode system 112. The impedance measurement module 402 may receive the signals S(1)-S(N) from the secondary electrode sets 502, determine localized impedance values Z(1)-Z(N), and provide the determined localized impedance values Z(1)-Z(N) to the secondary electrode contact determination module 404 at the distal portion 108. Additionally, at the distal portion 108, the secondary electrode contact determination module 404 may determine secondary electrode contact results based on the localized impedance values Z(1)-Z(N), and transmit the secondary electrode contact results over the single or common transmission line 506 to remaining components of the controller 118 positioned proximal the elongate member 110. In response to receipt of the secondary electrode contact results, the remaining components of the controller 118 may process the secondary electrode contact results and take further action as previously described with reference to FIG. 4.

For the configuration in FIG. 5D, the impedance measurement module 402, the secondary electrode contact determination module 404, and the primary electrode contact determination module 406 are included as part of the distal portion 108, such as part of the electrode system 112. The impedance measurement module 402 may receive the signals S(1)-S(N) from the secondary electrode sets 502, determine localized impedance values Z(1)-Z(N), and provide the determined localized impedance values Z(1)-Z(N) to the secondary electrode contact determination module 404 at the distal portion 108. Additionally, at the distal portion 108, the secondary electrode contact determination module 404 may determine secondary electrode contact results based on the localized impedance values Z(1)-Z(N), and provide the secondary electrode contact results to the primary electrode contact determination module 406. In response, the primary electrode contact determination module 406 may determine a primary electrode contact result, and transmit the primary electrode contact result over the single or common transmission line 506 to remaining components of the controller 118 positioned proximal the elongate member 110. In response to receipt of the secondary electrode contact results, the remaining components of the controller 118 may process the primary electrode contact result and take further action as previously described with reference to FIG. 4.

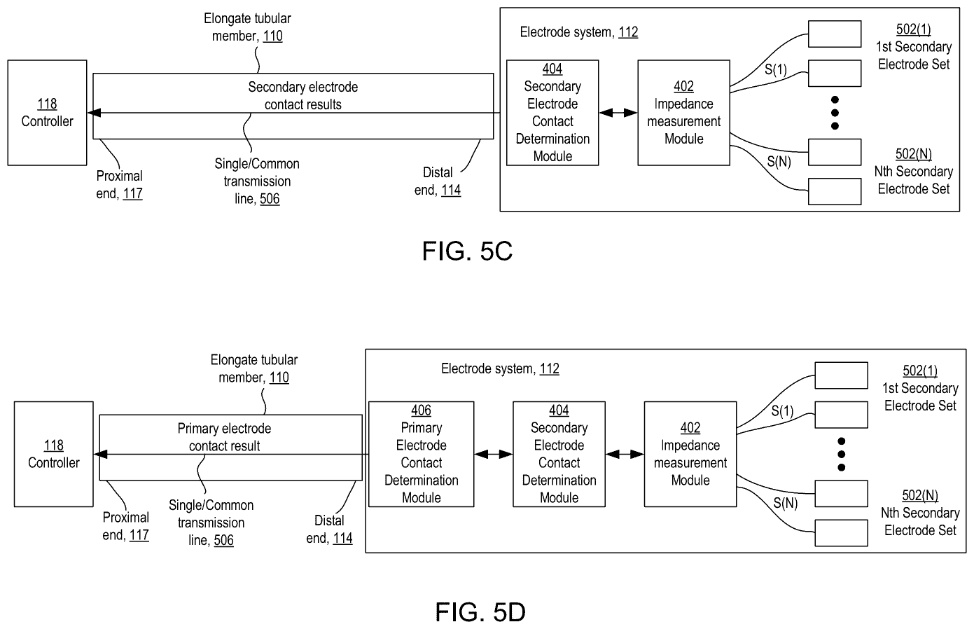

FIG. 6 shows a flow chart of an example method 600 for operating the electrosurgical device 102. The method 600 is described with reference to the various components of the electrosurgical device 102 described with reference to FIGS. 1-5D. At block 602, the controller 118 may receive at least one signal indicative of a plurality of localized impedances sensed by the secondary electrode assembly 304, such as by a plurality of periphery secondary electrode sets 304(P) and/or at least one core secondary electrode set 304(C). The plurality of localized impedances may be for the periphery 308 of the primary electrode assembly 302 and/or the core 322 of the primary electrode assembly 302. For at least some example methods, the controller 118 may bias or subject the secondary electrode assembly with a voltage, and the at least one signal it receives may be a response of the secondary electrode assembly to the voltage.

At block 604, the controller 118 may permit delivery of electrosurgical energy to the primary electrode assembly 302 in response to a determination that the primary electrode assembly 302 is sufficiently contacting tissue based on the plurality of localized impedances. In some example methods, the determination may be made internally by the controller 118. In other example methods, the determination may be made externally from the controller 118, such as by an external computing device or an operator. In addition, in some example methods, the controller 118 may directly or automatically control the delivery of electrosurgical energy to the primary electrode assembly 302 without external intervention. For example, the controller 118 may determine that the primary electrode assembly 302 is sufficiently contacting tissue, and in response, permit delivery of electrosurgical energy to the primary electrode assembly 302. In other methods, the controller 118 may control the delivery of electrosurgical energy with external intervention. For example, an external computing device or a person may determine whether the primary electrode assembly 302 is sufficiently contacting the tissue based on sensed localized impedances, and may control, command or instruct the controller 118 to permit the delivery of electrosurgical energy to the primary electrode assembly 302.

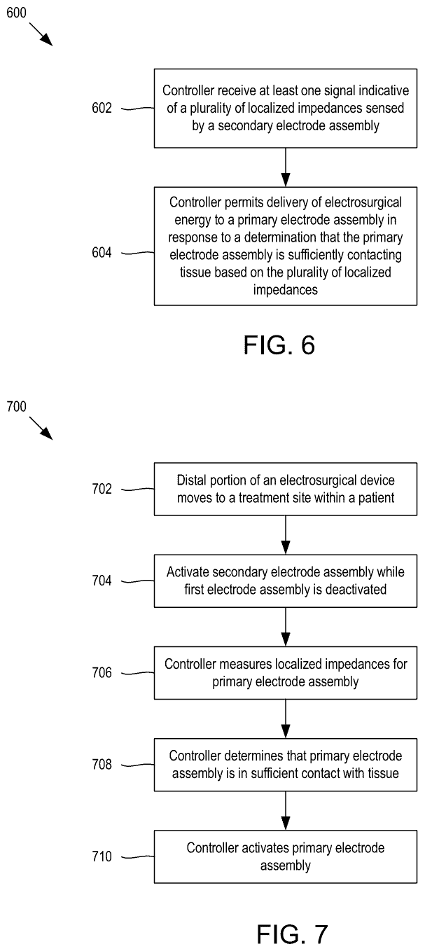

FIG. 7 is a flow chart of another example method 700 of operating the electrosurgical device 102. The method 600 is described with reference to the various components of the electrosurgical device 102 described with reference to FIGS. 1-5D. At block 702, the distal portion 108 of the electro surgical device 102 may move to a treatment site within a patient. The treatment site may include tissue that is to be subject to an electrosurgical effect, such as ablation, coagulation, or cutting. At block 704, the secondary electrode assembly 304 may be activated while the primary electrode assembly 302 is deactivated. For example, the controller 118, such as with the power source 104, may activate the secondary electrode assembly 304 by applying a voltage to secondary electrode sets of the secondary electrode assembly 304, which may cause the secondary electrode sets to draw respective electrical currents, amounts of which may indicate whether or not the secondary electrode sets are contacting the tissue at the treatment site.

At block 706, the controller 118 may measure, such as with the impedance measurement module 402, a plurality of localized impedances for the periphery 308 and/or the core 322 of the primary electrode assembly 302 in response to activating the secondary electrode assembly 304. At block 708, the controller may determine that the primary electrode assembly 302 is in sufficient contact with the tissue at the treatment site based on the plurality of localized impedances. For example, the controller 118 may determine, such as with secondary electrode contact determination module 404, a plurality of secondary electrode contact results based on the localized impedances values determined at block 706, and then determine, such as with a primary electrode contact determination module, a primary electrode contact result based on the plurality of secondary electrode contact results. For example, the controller 118 may determine that the secondary electrode contact results indicate that at least a predetermined number of secondary electrode sets is in contact with, or in sufficient contact with, the tissue, as previously described with reference to FIG. 4. At block 710, the controller 118 may activate the primary electrode assembly 302 in response to determining that the primary electrode assembly 302 is in contact with, or in sufficient contact with, the tissue. As previously described, the controller 118 may activate the primary electrode assembly 302 may permitting electrosurgical energy to be delivered to the primary electrode assembly 302 to cause an electrosurgical effect.

Methods other than methods 600 and 700 described with reference to FIGS. 6 and 7, respectively, may be possible, including methods performing any of the various actions by any of the various components of the electrosurgical system 100 described with reference to FIGS. 1-5D.

Additionally, the configurations described with reference to FIGS. 1-3 show the electrode system 112, the at least one electrode 202, the primary electrode assembly 302 and the secondary electrode assembly 304 configured generally as flat structures disposed on a generally flat substrate 204. In other configurations, including those intended for the electrodes to make contact to curved anatomical structures, the electrodes and/or the substrate(s) on which the electrodes are disposed by be curved structures. In addition or alternatively, for luminal application (e.g., the esophagus), the electrode system 112 may be mounted on the surface of an expandable or inflatable structure, such as a balloon. For configurations that use curved and/or expandable elements for incorporation with the electrode system 112 may be possible.

The subject matter of the present description may also relate, among others, to the following aspects:

A first aspect includes an electrosurgical device that includes: an elongate member extending from a proximal portion to a distal portion; a primary electrode assembly at the distal portion, the primary electrode assembly configured to contact tissue for performance of an electrosurgical procedure; and a secondary electrode assembly comprising a plurality of periphery secondary electrode sets, each periphery secondary electrode set configured to sense a respective one of a plurality of localized impedances for a periphery of the primary electrode assembly.

A second aspect includes the first aspect, and further includes wherein a number of the plurality of periphery secondary electrode sets is at least three.

A third aspect includes the second aspect, and further includes wherein the number is at least four.

A fourth aspect includes any of the first through third aspects, and further includes wherein at least one of the plurality of periphery secondary electrode sets is disposed outside of an outer boundary of the primary electrode assembly.

A fifth aspect includes the fourth aspect, and further includes wherein all of the plurality of periphery secondary electrode sets are disposed outside of the outer boundary of the primary electrode assembly.

A sixth aspect includes any of the first through fifth aspects, and further includes wherein the secondary electrode assembly further comprises a core secondary electrode set configured to sense a localized impedance for a core of the primary electrode assembly.

A seventh aspect includes any of the first through sixth aspects, and further includes wherein a periphery secondary electrode set of the plurality of periphery secondary electrode sets is disposed adjacent to a corner of an outer boundary of the primary electrode assembly.

An eighth aspect includes the seventh aspect, and further includes wherein at least two periphery secondary electrode sets of the plurality of periphery secondary electrode sets are disposed adjacent to at least two corners of the outer boundary.

A ninth aspect includes the eighth aspect, and further includes wherein the plurality of periphery secondary electrode sets comprises four periphery secondary electrode sets, and the outer boundary comprises four corners, and wherein each of the four periphery secondary electrode sets is disposed adjacent to a respective one of the four corners.

A tenth aspect includes any of the first through ninth aspects, and further includes wherein a periphery secondary electrode set of the plurality of periphery secondary electrode sets is disposed adjacent to a side of an outer boundary of the primary electrode assembly.

An eleventh aspect includes the tenth aspect, and further includes wherein at least two periphery secondary electrode sets of the plurality of periphery secondary electrode sets are disposed adjacent to at least two sides of the outer boundary.

A twelfth aspect includes the eleventh aspect, and further includes wherein the plurality of periphery secondary electrode sets comprises four periphery secondary electrode sets, and the outer boundary comprises four sides, and wherein each of the four periphery secondary electrode sets is disposed adjacent to a respective one of the four sides.

A thirteenth aspect includes any of the first through twelfth aspects, and further includes wherein a first periphery secondary electrode set of the plurality of electrode sets is disposed adjacent to a corner of an outer boundary of the primary electrode assembly, and a second periphery secondary electrode set is disposed adjacent to a side of the outer boundary.

A fourteenth aspect includes any of the first through thirteenth aspects, and further includes wherein an outer boundary of the primary electrode assembly is a polygonal shape.

A fifteenth aspect includes the fourteenth aspect, and further includes wherein the polygonal shape comprises a rectangle.

A sixteenth aspect includes any of the first through fifteenth aspects, and further includes wherein the primary electrode assembly and the secondary electrode assembly are disposed on a same substrate.

A seventeenth aspect includes any of the first through sixteenth aspects, and further includes wherein the secondary electrode assembly is configured to be electrically activated independent of the primary electrode assembly.

An eighteenth aspect includes any of the first through seventeenth aspects, and further includes wherein an overall surface area of the primary electrode assembly is at least twice an overall surface area of the secondary electrode assembly.

A nineteenth aspect includes the eighteenth aspect, and further includes wherein the overall surface area of the primary electrode is at least ten times the overall surface area of the secondary electrode assembly.

A twentieth aspect includes an electrosurgical system that includes: a controller configured to: receive at least one signal indicative of a plurality of localized impedances for a periphery of a primary electrode assembly configured to cause an electrosurgical effect on tissue at a treatment site within a patient, the plurality of localized impedances sensed by a secondary electrode assembly; and permit delivery of electrosurgical energy to the primary electrode assembly in response to a determination that the primary electrode assembly is sufficiently contacting the tissue based on the plurality of localized impedances sensed by the secondary electrode assembly.

A twenty-first aspect includes the twentieth aspect, and further includes wherein the controller is further configured to output, via an output device, the plurality of localized impedances.

A twenty-second aspect includes the twentieth or twenty-first aspect, and further includes wherein the controller is further configured to output, via an output, an indication that indicates whether the primary electrode assembly is sufficiently in contact with the tissue.

A twenty-third aspect includes any of the twentieth through twenty-second aspects, and further includes wherein the controller is further configured to determine that the primary electrode assembly is in sufficient contact with the tissue based on the plurality of localized impedances.

A twenty-fourth aspect includes any of the twentieth through twenty-third aspects, and further includes wherein the controller is further configured to: determine whether each of a plurality of periphery secondary electrode sets of the secondary electrode assembly is in sufficient contact with the tissue based on the plurality of localized impedances; determine whether a predetermined amount of the plurality of periphery secondary electrode sets is in sufficient contact with the tissue; and determine that the primary electrode assembly is in sufficient contact with the tissue in response to the predetermined number of the plurality of periphery secondary electrode sets being in sufficient contact with the tissue.

A twenty-fifth aspect includes the twenty-fourth aspect, and further includes wherein the predetermined amount of the plurality of periphery secondary electrode sets comprises all of the plurality of periphery secondary electrode sets.

A twenty-sixth aspect includes any of the twentieth through twenty-fifth aspects, and further includes a single transmission line longitudinally extending from a proximal portion to a distal portion of an elongate member, the single transmission line configured to transmit at least one of: the plurality of localized impedances, a plurality of contact results indicating whether each of the plurality of periphery secondary electrode sets is in sufficient contact with the tissue, or a contact result indicating whether the primary electrode assembly is in sufficient contact with the tissue.

A twenty-seventh aspect includes any of the twentieth through twenty-sixth aspects, and further includes wherein the controller is further configured to simultaneously permit delivery of the electrosurgical energy to the primary electrode assembly and activate the secondary electrode assembly to sense the plurality of localized impedances.

A twenty-eighth aspect includes the twenty-seventh aspect, and further includes that the controller is further configured to determine whether the primary electrode assembly is in sufficient contact with the tissue based on the plurality of localized impedances while permitting the delivery of the electrosurgical energy to the primary electrode assembly.

A twenty-ninth aspect includes any of the twentieth through twenty-eighth aspects, and further includes that the electrosurgical system further includes the electrosurgical device of any of the first through nineteenth aspects.

A thirtieth aspect includes a method for electrosurgery, the method including: moving a distal portion of an electrosurgical device to a treatment site within a patient, the distal portion comprising a primary electrode assembly and a secondary electrode assembly; while the primary electrode assembly is deactivated, activating the secondary electrode assembly; in response to activating the secondary electrode assembly, measuring, with a controller, a plurality of localized impedances for a periphery of the primary electrode assembly; determining, with the controller, that the primary electrode assembly is in sufficient contact with tissue at the treatment site based on the plurality of localized impedances; and in response to determining that the primary electrode assembly is in sufficient contact with the tissue at the treatment site, activating the primary electrode assembly with electrosurgical energy to cause an electrosurgical effect on the tissue.

A thirty-first aspect includes the thirtieth aspect, and further includes: outputting, with an output device, the plurality of localized impedances.

A thirty-second aspect includes the thirtieth or thirty-first aspects, and further includes: outputting, with an output device, an indication that indicates whether the primary electrode assembly is sufficiently in contact with the tissue.

A thirty-third aspect includes any of the thirtieth through thirty-second aspects, and further includes: determining, with the controller, whether each of a plurality of periphery secondary electrode sets of the secondary electrode assembly is in sufficient contact with the tissue based on the plurality of localized impedances; determining, with the controller, whether a predetermined number of the plurality of periphery secondary electrode sets is in sufficient contact with the tissue; and determining, with the controller, that the primary electrode assembly is in sufficient contact with the tissue in response to determining that the predetermined number of the plurality of periphery secondary electrode sets is in sufficient contact with the tissue.

A thirty-fourth aspect includes the thirty-third aspect, and further includes wherein the predetermined amount of the plurality of periphery secondary electrode sets comprises all of the plurality of periphery secondary electrode sets.

A thirty-fifth aspect includes any of the thirtieth through thirty-fourth aspects, and further includes: transmitting, with a single transmission line longitudinally extending from a proximal portion to a distal portion of an elongate member, at least one of: the plurality of localized impedances, a plurality of contact results indicating whether each of the plurality of periphery secondary electrode sets is in sufficient contact with the tissue, or a contact result indicating whether the primary electrode assembly is in sufficient contact with the tissue.

A thirty-sixth aspect includes any of the thirtieth through thirty-fifth aspects, and further includes: simultaneously activating, with the controller, the primary electrode assembly with the electrosurgical energy and the secondary electrode assembly to sense the plurality of localized impedances.

A thirty-seventh aspect includes the thirty-sixth aspect, and further includes: determining, with the controller, whether the primary electrode assembly is in sufficient contact with the tissue based on the plurality of localized impedances while activating the primary electrode assembly with the electrosurgical energy.

The foregoing description of various embodiments of the invention has been presented for purposes of illustration and description. It is not intended to be exhaustive or to limit the invention to the precise embodiments disclosed. Numerous modifications or variations are possible in light of the above teachings. The embodiments discussed were chosen and described to provide the best illustration of the principles of the invention and its practical application to thereby enable one of ordinary skill in the art to utilize the invention in various embodiments and with various modifications as are suited to the particular use contemplated. All such modifications and variations are within the scope of the invention as determined by the appended claims when interpreted in accordance with the breadth to which they are fairly, legally, and equitably entitled.

1. An electrosurgical device comprising:
an elongate member extending from a proximal portion to a distal portion;
a primary electrode assembly at the distal portion, the primary electrode assembly configured to contact tissue for performance of an electrosurgical procedure; and
a secondary electrode assembly comprising a plurality of periphery secondary electrode sets, each periphery secondary electrode set configured to sense a respective one of a plurality of localized impedances for a periphery of the primary electrode assembly.

2. The electrosurgical device of claim 1, wherein a number of the plurality of periphery secondary electrode sets is at least three.

3. The electrosurgical device of claim 2, wherein the number is at least four.

4. The electrosurgical device of claim 1, wherein at least one of the plurality of periphery secondary electrode sets is disposed outside of an outer boundary of the primary electrode assembly.

5. The electrosurgical device of claim 1, wherein the secondary electrode assembly further comprises a core secondary electrode set configured to sense a localized impedance for a core of the primary electrode assembly.

6. The electrosurgical device of claim 1, wherein a periphery secondary electrode set of the plurality of periphery secondary electrode sets is disposed adjacent to a corner of an outer boundary of the primary electrode assembly.

7. The electrosurgical device of claim of 1, wherein a periphery secondary electrode set of the plurality of periphery secondary electrode sets is disposed adjacent to a side of an outer boundary of the primary electrode assembly.

8. The electrosurgical device of claim 1, wherein an outer boundary of the primary electrode assembly is a polygonal shape.

9. The electrosurgical device of claim 1, wherein the secondary electrode assembly is configured to be electrically activated independent of the primary electrode assembly.

10. The electrosurgical device of claim 1, wherein an overall surface area of the primary electrode assembly is at least twice an overall surface area of the secondary electrode assembly.

11. An electrosurgical system comprising:
a controller configured to:
receive at least one signal indicative of a plurality of localized impedances for a periphery of a primary electrode assembly configured to cause an electrosurgical effect on tissue at a treatment site within a patient, the plurality of localized impedances sensed by a secondary electrode assembly; and
permit delivery of electrosurgical energy to the primary electrode assembly in response to a determination that the primary electrode assembly is sufficiently contacting the tissue based on the plurality of localized impedances sensed by the secondary electrode assembly.

12. The electrosurgical system of claim 11, wherein the controller is further configured to output, via an output device, the plurality of localized impedances.

13. The electrosurgical system of claim 11, wherein the controller is further configured to output, via an output, an indication that indicates whether the primary electrode assembly is sufficiently in contact with the tissue.

14. The electrosurgical system of claim 11, wherein the controller is further configured to determine that the primary electrode assembly is in sufficient contact with the tissue based on the plurality of localized impedances.

15. The electrosurgical system of claim 11, wherein the controller is further configured to:
determine whether each of a plurality of periphery secondary electrode sets of the secondary electrode assembly is in sufficient contact with the tissue based on the plurality of localized impedances;
determine whether a predetermined amount of the plurality of periphery secondary electrode sets is in sufficient contact with the tissue; and
determine that the primary electrode assembly is in sufficient contact with the tissue in response to the predetermined number of the plurality of periphery secondary electrode sets being in sufficient contact with the tissue.

16. The electrosurgical system of claim 15, wherein the predetermined amount of the plurality of periphery secondary electrode sets comprises all of the plurality of periphery secondary electrode sets.

17. The electrosurgical system of claim 11, further comprising a single transmission line longitudinally extending from a proximal portion to a distal portion of an elongate member, the single transmission line configured to transmit at least one of: the plurality of localized impedances, a plurality of contact results indicating whether each of the plurality of periphery secondary electrode sets is in sufficient contact with the tissue, or a contact result indicating whether the primary electrode assembly is in sufficient contact with the tissue.

18. The electrosurgical system of claim 11, wherein the controller is further configured to simultaneously permit delivery of the electrosurgical energy to the primary electrode assembly and activate the secondary electrode assembly to sense the plurality of localized impedances.

19. The electrosurgical system of claim 18, wherein the controller is further configured to determine whether the primary electrode assembly is in sufficient contact with the tissue based on the plurality of localized impedances while permitting the delivery of the electrosurgical energy to the primary electrode assembly.

20. A method for electrosurgery, the method comprising:
moving a distal portion of an electrosurgical device to a treatment site within a patient, the distal portion comprising a primary electrode assembly and a secondary electrode assembly;
while the primary electrode assembly is deactivated, activating the secondary electrode assembly;
in response to activating the secondary electrode assembly, measuring, with a controller, a plurality of localized impedances for a periphery of the primary electrode assembly;
determining, with the controller, that the primary electrode assembly is in sufficient contact with tissue at the treatment site based on the plurality of localized impedances; and
in response to determining that the primary electrode assembly is in sufficient contact with the tissue at the treatment site, activating the primary electrode assembly with electrosurgical energy to cause an electrosurgical effect on the tissue.
